(19) United States
(12) Patent Application Publication
(10) Pub. No.: US 2016/0317075 A1
(43) Pub. Date: Nov. 3, 2016

(54) Novel diagnostic algorithm for acute kidney injury in hospitalized children

(71) Applicant: The Board of Trustees of the Leland Stanford, Junior, University, Palo Alto, CA (US)

(72) Inventors: Jun Ji, Oingdao (CN); Bruce Xuefeng Ling, Palo Alto, CA (US); Scott Sutherland, Palo Alto, CA (US)

(21) Appl. No.: 14/485,575

(22) Filed: Sep. 12, 2014

Related U.S. Application Data
(60) Provisional application No. 61/876,763, filed on Sep. 12, 2013.

(51) Int. Cl. A61B 5/20; G06F 19/00; A61B 5/00

(52) U.S. Cl. CPC A61B 5/201; A61B 5/4842; A61B 5/7275; G06F 19/345

(57) ABSTRACT

We have developed a novel AKI diagnostic algorithm upon KID 2009 database. The KID is multi-featured and the AKI and non-AKI groups are highly imbalanced, making it challenging to describe them via simple linear statistics. Thus, to identify features effectively, our AKI association studies employed statistical learning strategies; a predictive model was created to accurately determine which KID data elements were highly associated with an AKI diagnosis. We employed prediction analysis of microarrays (PAM), which is commonly applied to high-feature datasets such as DNA microarrays; PAM determines which data elements, or features, best contribute to the predictive model or characterize individual classes/cohorts, Clinical Classification Software codes (286 diagnosis, 231 procedural) were used to bin ICD-9-CM codes (n=6,722) and analyzed by PAM. PAM identified relevant AKI predictors and eliminated irrelevant data elements, which constitute noise.

### FIELD OF THE INVENTION

The present invention generally relates to the field of medical diagnosis of kidney injuries.

### BACKGROUND OF THE INVENTION

Acute kidney injury (AKI), which can be an abrupt decline in renal function, is a common complication amongst hospitalized patients with a rising incidence. Although AKI is common amongst hospitalized children, comprehensive epidemiologic data are lacking

There is a need in the art for improved methods for diagnosing kidney health including methods for diagnosing acute kidney injury in children and adults.

### SUMMARY OF THE INVENTION

We have developed a novel AKI diagnostic algorithm upon KID 2009 database. The KID is multi-featured and the AKI and non-AKI groups are highly imbalanced, making it challenging to describe them via simple linear statistics. Thus, to identify features effectively, our AKI association studies employed statistical learning strategies; a predictive model was created to accurately determine which KID data elements were highly associated with an AKI diagnosis. We employed prediction analysis of microarrays (PAM), which is commonly applied to high-feature datasets such as DNA microarrays; PAM determines which data elements, or features, best contribute to the predictive model or characterize individual classes/cohorts, Clinical Classification Software codes (286 diagnosis, 231 procedural) were used to bin ICD-9-CM codes (n=6,722) and analyzed by PAM. PAM identified relevant AKI predictors and eliminated irrelevant data elements, which constitute noise.

Subsequently, the data was subjected to machine learning/pattern recognition predictive modeling analyses using linear discriminant analysis (LDA). LDA maximizes the ratio of between-class variance to within-class variance, guaranteeing maximal separation between the AKI and non-AKI classes. At the outset, the data were randomly divided into a training dataset (⅔ of the records) and a testing/validation dataset (⅓ of the records); the training data were used to design the prediction model and the testing/validation data were used to confirm its accuracy. The results of this analysis are presented as unadjusted odds ratios (OR). Notably, the datasets are classimbalanced since one class (non-AKI) contains significantly more subjects than the other (AKI). Thus, repeated bootstrapping (n=100) was integrated with a voting mechanism to derive the final classification result. ROC analysis was performed to evaluate the performance of the model. Area under the ROC curve was calculated using ROCR package. Odds ratios of the PAM selected features were computed using generalized linear modeling method.

Of 2,644,263 children, 10,322 developed AKI (3.9 per 1000 admissions). Although 19% of the AKI cohort was 1 mo, the highest incidence was seen in children 15-18 y (6.6 per 1000 admissions). 49% of the AKI cohort was White, however, AKI incidence was higher amongst African- Americans (4.5 vs. 3.8 per 1000 admissions). In-hospital mortality amongst patients with AKI was 15.3%, but higher amongst children 1 mo (31.3% vs. 10.1%, p&lt;0.001) and those requiring critical care (32.8% vs. 9.4%, p&lt;0.001) or dialysis (27.1% vs. 14.2%, p&lt;0.001). Shock (OR 2.15, 95% CI 1.95-2.36), septicemia (OR 1.37, 95% CI 1.32-1.43), intubation/mechanical ventilation (OR 1.2, 95% CI 1.16-1.25), circulatory disease (OR 1.47, 95% CI 1.32-1.65), cardiac congenital anomalies (OR 1.2, 95% CI 1.13-1.23), and extracorporeal support (OR 2.58, 95% CI 2.04-3.26) were associated with AKI. The overall predictive model for AKI in hospitalizations among children &lt;=1 month and &gt; 1 month of age resulted in ROC AUCs of 0.94 and 0.98, respectively.

These and other embodiments and advantages can be more fully appreciated upon an understanding of the detailed description of the invention as disclosed below in conjunction with the attached Figures.

### BRIEF DESCRIPTION OF THE DRAWINGS

The following drawings will be used to more fully describe embodiments of the present invention.

FIG. 1 is a block diagram of a computer system on which the present invention can be implemented.

### DETAILED DESCRIPTION OF THE INVENTION

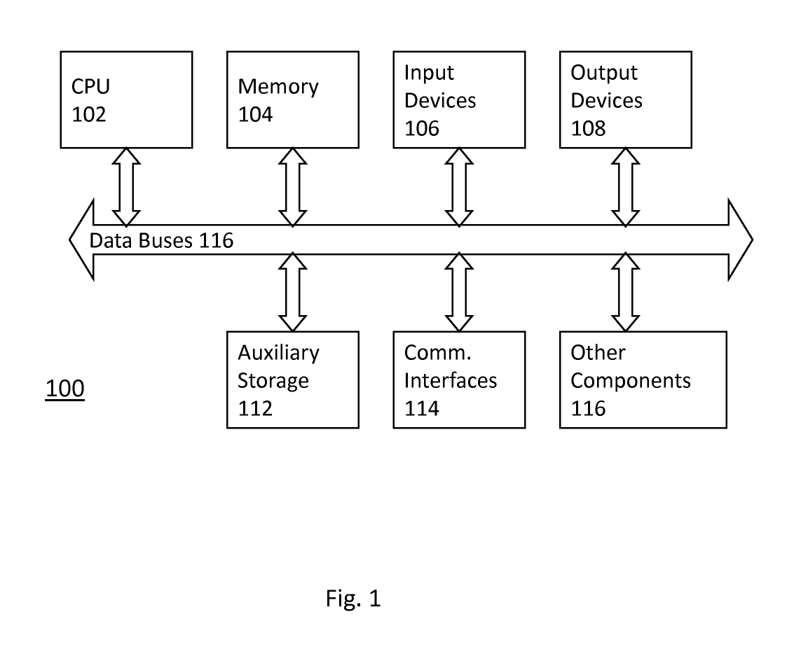

Among other things, the present invention relates to methods, techniques, and algorithms that are intended to be implemented in a digital computer system 100 such as generally shown in FIG. 1. Such a digital computer is well-known in the art and may include the following.

Computer system 100 may include at least one central processing unit 102 but may include many processors or processing cores. Computer system 100 may further include memory 104 in different forms such as RAM, ROM, hard disk, optical drives, and removable drives that may further include drive controllers and other hardware.

Auxiliary storage 112 may also be include that can be similar to memory 104 but may be more remotely incorporated such as in a distributed computer system with distributed memory capabilities.

Computer system 100 may further include at least one output device 108 such as a display unit, video hardware, or other peripherals (e.g., printer). At least one input device 106 may also be included in computer system 100 that may include a pointing device (e.g., mouse), a text input device (e.g., keyboard), or touch screen.

Communications interfaces 114 also form an important aspect of computer system 100 especially where computer system 100 is deployed as a distributed computer system. Computer interfaces 114 may include LAN network adapters, WAN network adapters, wireless interfaces, Bluetooth interfaces, modems and other networking interfaces as currently available and as may be developed in the future.

Computer system 100 may further include other components 116 that may be generally available components as well as specially developed components for implementation of the present invention. Importantly, computer system 100 incorporates various data buses 116 that are intended to allow for communication of the various components of computer system 100. Data buses 116 include, for example, input/output buses and bus controllers.

Indeed, the present invention is not limited to computer system 100 as known at the time of the invention. Instead, the present invention is intended to be deployed in future computer systems with more advanced technology that can make use of all aspects of the present invention. It is expected that computer technology will continue to advance but one of ordinary skill in the art will be able to take the present disclosure and implement the described teachings on the more advanced computers or other digital devices such as mobile telephones or “smart” televisions as they become available. Moreover, the present invention may be implemented on one or more distributed computers. Still further, the present invention may be implemented in various types of software languages including C, C++, and others. Also, one of ordinary skill in the art is familiar with compiling software source code into executable software that may be stored in various forms and in various media (e.g., magnetic, optical, solid state, etc.). One of ordinary skill in the art is familiar with the use of computers and software languages and, with an understanding of the present disclosure, will be able to implement the present teachings for use on a wide variety of computers.

The present disclosure provides a detailed explanation of the present invention with detailed explanations that allow one of ordinary skill in the art to implement the present invention into a computerized method. Certain of these and other details are not included in the present disclosure so as not to detract from the teachings presented herein but it is understood that one of ordinary skill in the art would be familiar with such details.

Further details of the present invention are included in the Appendix which is herein incorporated by reference for all purposes.

It should be appreciated by those skilled in the art that the specific embodiments disclosed herein may be readily utilized as a basis for modifying or designing other algorithms or systems. It should also be appreciated by those skilled in the art that such modifications do not depart from the scope of the invention as set forth in the appended claims. For example, variations to the methods can include changes that may improve the accuracy or flexibility of the disclosed methods.

What is claimed is:

1. A computerized method for patient diagnosis, comprising:
receiving medical information for individuals with acute kidney injury;
receiving medical information for individuals without acute kidney injury;
applying statistical learning methods to the highly imbalanced dataset to derive AKI-related risk factors.
